(19) United States
(12) Patent Application Publication
(10) Pub. No.: US 2024/0420849 A1
(43) Pub. Date: Dec. 19, 2024

(54) ELECTRONIC HEALTH RECORDS DATA SUMMARIZATION FOR GRAPH MACHINE LEARNING

(71) Applicant: Accenture Global Solutions Limited, Dublin (IE)

(72) Inventors: Christophe Dominique Marie Gueret, Dublin (IE); Luca Costabello, Newbridge (IE); Jeffrey Sardina, Mhic an Bhaird (IE)

(21) Appl. No.: 18/333,875

(22) Filed: Jun. 13, 2023

(51) Int. Cl. G16H 50/70; G16H 10/60

(52) U.S. Cl. CPC G16H 50/70; G16H 10/60

(57) ABSTRACT

Implementations include actions of receiving EHR data for a set of patients, defining a set of patient groups from the EHR data that is representative of a subset of patients, the set of patient groups being defined using a set of criteria, generating, for each patient group, a set of demographics triples and a set of medical triples, demographics triples including one or more links between patient groups and one or more demographics entities, and medical triples in the set of medical triples including one or more links between patient groups and one or more medical entities, providing a patients graph using the set of demographics triples and the set of medical triples, training a KGE model using a KG and the patients graph to provide a trained KGE model, and providing the trained KGE model for inference to predict likelihood that a link between entities is factually correct.

### BACKGROUND

Knowledge graphs are graph datasets that represent networks of real-world entities and characterizes the relationships between them. Example entities include objects, events, situations, or concepts. Knowledge graphs include a set of nodes and a set of edges, where the nodes represent respective entities and edges between nodes define relationships between the nodes.

Graph machine learning is a family of machine learning methods designed to learn from graph datasets with the goal of inferring missing information (e.g., predicting missing edges between nodes of a graph). Graph machine learning includes node representation learning models based on graph features, graph neural networks, and neural link predictors. A neural link predictor (also referred to as knowledge graph embedding (KGE) model) is an artificial neural network architecture that learns vector representations (referred to as “embeddings”) of concepts by a training a knowledge graph to predict missing, unseen links between nodes.

### SUMMARY

Implementations of the present disclosure are generally directed to graph machine learning (GML). More particularly, implementations of the present disclosure are directed to provisioning datasets that are optimized for training GML models using electronic health record (EHR) data from clinical systems.

In some implementations, actions include receiving electronic health record (EHR) data including medical records for a set of patients, defining a set of patient groups from a subset of the EHR data that is representative of a subset of patients of the set of patients, the set of patient groups being defined using a set of criteria providing within a grouping strategy, generating, for each patient group in the set of patient groups, a set of demographics triples and a set of medical triples, demographics triples in the set of demographics triples including one or more links between patient groups and one or more demographics entities, and medical triples in the set of medical triples including one or more links between patient groups and one or more medical entities, providing a patients graph using the set of demographics triples and the set of medical triples, training a KGE model using a KG and the patients graph to provide a trained KGE model, and providing the trained KGE model for inference to predict likelihood that a link between entities is factually correct. Other implementations of this aspect include corresponding systems, apparatus, and computer programs, configured to perform the actions of the methods, encoded on computer storage devices.

These and other implementations can each optionally include one or more of the following features: actions further include extracting the subset of the EHR data from the EHR data using a cohort definition that defines at least one cohort criterio; generating the set of medical triples includes determining a set of risk factors that represent relative risk of co-occurrence of conditions within the subset of EHR data, and for each risk factor that meets a threshold risk factor, creating a medical triple representative of the conditions in the set of medical triples; a sampling strategy is used to determine the threshold risk factor; one or more medical triples represent at least one drug relative to one or more patient groups, the at least one drug being selected for inclusion in the medical triple based on a head-tail analysis of a drug statistics determined from the EHR data based on the patient groups; providing a patients graph using the set of demographics triples and the set of medical triples includes concatenating demographics triples and medical triples; and demographics triples in the set of demographics triples and medical triples in the set of medical triples are generated based on a target ontology.

The present disclosure also provides a computer-readable storage medium coupled to one or more processors and having instructions stored thereon which, when executed by the one or more processors, cause the one or more processors to perform operations in accordance with implementations of the methods provided herein.

The present disclosure further provides a system for implementing the methods provided herein. The system includes one or more processors, and a computer-readable storage medium coupled to the one or more processors having instructions stored thereon which, when executed by the one or more processors, cause the one or more processors to perform operations in accordance with implementations of the methods provided herein.

It is appreciated that methods in accordance with the present disclosure can include any combination of the aspects and features described herein. That is, for example, apparatus and methods in accordance with the present disclosure are not limited to the combinations of aspects and features specifically described herein, but also may include any combination of the aspects and features provided.

The details of one or more implementations of the present disclosure are set forth in the accompanying drawings and the description below. Other features and advantages of the present disclosure will be apparent from the description, drawings, and claims.

### BRIEF DESCRIPTION OF DRAWINGS

FIG. 1 depicts an example system that can execute implementations of the present disclosure.

FIG. 2 depicts an example portion of an example knowledge graph.

FIG. 3 depicts a conceptual architecture for electronic health record data summarization in accordance with implementations of the present disclosure.

FIG. 4 depicts example group graphs representing example demographics triples in accordance with implementations of the present disclosure.

FIG. 5 depicts an example group graph representing example medical triples in accordance with implementations of the present disclosure.

FIG. 6 depicts an example head-tail graph for prescription drugs.

FIG. 7 depicts an example target ontology in accordance with implementations of the present disclosure.

FIG. 8 depicts an example process that can be executed in accordance with implementations of the present disclosure.

Like reference numbers and designations in the various drawings indicate like elements.

### DETAILED DESCRIPTION

Implementations of the present disclosure are generally directed to graph machine learning (GML). More particularly, implementations of the present disclosure are directed to provisioning datasets that are optimized for training GML models using electronic health record (EHR) data from clinical systems.

In some implementations, actions include receiving electronic health record (EHR) data including medical records for a set of patients, defining a set of patient groups from a subset of the EHR data that is representative of a subset of patients of the set of patients, the set of patient groups being defined using a set of criteria providing within a grouping strategy, generating, for each patient group in the set of patient groups, a set of demographics triples and a set of medical triples, demographics triples in the set of demographics triples including one or more links between patient groups and one or more demographics entities, and medical triples in the set of medical triples including one or more links between patient groups and one or more medical entities, providing a patients graph using the set of demographics triples and the set of medical triples, training a KGE model using a KG and the patients graph to provide a trained KGE model, and providing the trained KGE model for inference to predict likelihood that a link between entities is factually correct.

For purposes of non-limiting illustration, implementations of the present disclosure are described in further detail herein with reference to an example use case. The example use case includes provisioning GML models using optimized datasets that are generated using EHR data from clinical systems. In this example context, knowledge graphs (KGs) can be used to represent biomedical knowledge bases. It is contemplated, however, that implementations of the present disclosure can be used in any appropriate use case.

To provide context for implementations of the present disclosure, and as introduced above, KGs are graph datasets that represent networks of real-world entities and characterize the relationships between them. Example entities include objects, events, situations, or concepts. KGs include a set of nodes and a set of edges, where the nodes represent respective entities and edges between nodes define relationships between the nodes. More particularly, a KG can be described as a graph dataset made of directed, label edges that connect nodes representing concepts (e.g., patients, medical conditions, genes, proteins, etc). In some instances, two nodes can be connected by multiple edges with distinct labels. As a result, KGs are multi-relational graphs. KGs can be defined using triples, each triple being a fact (also referred to as link) that can be defined as t=(s, p, o), where s is a subject, p is a predicate, and o is an object. In the example use case, an example triple can be (Patient2, undergoneProcedure, Mastectomy), where Patient2 is a patient and Mastectomy is a surgical procedure.

GML is a family of machine learning methods designed to learn from graph datasets with a goal of inferring missing information (e.g., predicting missing edges between nodes of a graph). GML includes node representation learning models based on graph features, graph neural networks (GNNs), and neural link predictors. A neural link predictor (also referred to as KG embedding (KGE) model) is an artificial neural network architecture that learns vector representations (referred to as “embeddings”) of concepts by training using a KG (e.g., a training KG labeled as G) to predict missing, unseen links between nodes.

In some examples, the KGE model (neural link predictor) includes an input layer, a corruption generation layer, an embedding lookup layer, a scoring layer, and a loss layer. The KG is fed to the KGE model as sets of triples at the input layer in the form of triple. The corruption generation layer takes triples and generates corruptions (also referred to herein as synthetic negatives) by randomly replacing either the subject or object with a random entity from the KG. The synthetic negatives, which generally represent implausible facts (e.g., (Mastectomy, undergoneProcedure, Patient2)) are used during training of the KGE model.

In the context of the present disclosure, an embedding is a k-dimensional vector of real numbers that represents either a node (e.g., Mastectomy) or an edge type (e.g., hasComorbidity). Embeddings are learned by artificial neural networks (e.g., the KGE model) and serve as their internal representation for the concept learned from the KG. Embeddings learned by neural link predictors typically have dimensionality k, where 100≤k≤500. That is, each embedding can be an array of 100-500 real numbers. The embedding lookup layer determines the embeddings of the entities and predicates of the triples and corruptions.

The scoring layer takes in the embeddings of the triples and respective corruptions and provides a score using one or more scoring functions (e.g., TransE, ComplEx, DistMult, HolE). The higher the score, the higher the likelihood that the respective triple is factually correct. The scoring layer can be considered as a way to assign a plausibility score to each fact of the knowledge graph. Scoring functions can rely on different intuitions. For example, TransE relies on distances, DistMult and ComplEx are bilinear-diagonal models, and HolE uses circular correlation. The loss layer uses the scores of the triples and their respective corruptions and computes a loss value.

As discussed above, the KGE model can be used for a link prediction task, which can be described as the task of inferring whether a new link between two nodes (e.g., t′=(s′, p′, o′)) of the KG (G) is factually correct. The KGE model carries out such predictions by combining the embeddings of s′, p′ and o′ to return a probability r that t′ is correct.

Further detail on KGs, GML, and neural link predictors is provided in commonly assigned U.S. patent application Ser. No. 18/161,260, filed on Jan. 30, 2023, and entitled Ontology-Driven Parameter Efficient Representations for Knowledge Graphs, the disclosure of which is expressly incorporated herein by reference in the entirety for all purposes.

In a traditional approach, and with reference to the example use case, KGs can capture a combination of medical records data and background biology data. In some examples, medical records data can be recorded in an EHR (e.g., created based on the Fast Healthcare Interoperability Resources (FHIR) provided by HL7). In some examples, background biology data can be recorded in one or more background data sources (e.g., the chemical database of bioactive molecules (ChEMBL) and UniProt provided by the European Bioinformatics Institute (EBI)). For example, the medical records data and the background biology data can be input to a GML module that provides a (trained) KGE model. The KGE model generates a set of link hypotheses and ranks as output.

A target ontology can be used, which defines what concepts will be put into the KG and how the concepts are connected. This can work well for the background biology data, but presents challenges in the integration of the medical records from EHRs. More particularly, neural link predictors, such as the KGE model of the present disclosure, are designed to work with KGs that express factual knowledge (e.g., statements in the form of “patient X has condition Y”). EHRs, however, are record-oriented in a way that can cloud factual knowledge. For example, an example record from an EHR can include “Consultant Z diagnosed the condition Y for patient X.” While such records are more accurate from a data record perspective and may include more data about the diagnosis (e.g., date, place of examination), they include extra nodes in the KG (e.g., the diagnosis node) and can break direct links (e.g., the direct link between patient X and condition Y) in factual statements.

Accordingly, EHR data can include additional data (data verbosity) that obscures direct links between entities. For example, an example EHR ontology derived from FHIR can include a patient node, a diagnosis node, a condition node, a prescription node, and a medication node, in which there is no direct link between the patient node and the condition node or the patient node and the medication node. Instead, in this example, the patient node is indirectly linked to the condition node through the diagnosis node and the prescription node (among other nodes). Also in this example, the patient node is indirectly linked to the medication node through the prescription node. Here, the diagnosis node and the prescription node, among others, are noise for the KGE model getting in the way of informing the KGE model about the condition of a patient and the drugs taken by this patient. Instead, with this extra data, the KGE model will learn how to predict new links between a diagnosis and a condition, but not between a patient and a condition. Although this may be useful for use-cases where the aim is to complete a previous diagnosis where conditions are suspected to be missing (or mis-diagnosed) this is less pertinent for more general use-cases.

It is however not as easy as just removing the intermediate nodes (e.g., diagnosis, prescription), because such intermediate nodes provide important information about conditions diagnosed at the same time and being thus potential co-morbidities, for example. That is, removing intermediate nodes results in a loss of information that is still required for training the KGE model. Further, this problem of data verbosity in the EHR data is detrimental to performance on link prediction tasks (e.g., between genes and diseases) where the EHR data is used only as background knowledge-devoid from links being directly predicted.

Accordingly, and in the example use case, implementations of the present disclosure are directed to, for example, generating a derivative dataset, also referred to as an optimized dataset, from EHR data that reduces data verbosity and surfaces direct links, while preserving, for example, co-diagnosis information, among other types of information. Further, implementations of the present disclosure enable certain entities (e.g., co-drugs) to be taken out, which are irrelevant to other entities of interest (e.g., the condition(s)). Also, implementations of the present disclosure mitigate imbalances created by having multiples of the same types of entities (e.g., patient nodes) pointing to one of another type of entity (e.g., condition). These imbalances result from EHR data being extracted for a cohort (e.g., a set of patients all having one condition in common and maybe other shared demographics). Neural link predictors are known to not deal well with such imbalances.

In accordance with implementations of the present disclosure, and as described in further detail herein, the optimized dataset is provided based on personas, also referred to as patient groups herein. Through use of patient groups, implementations of the present disclosure control part of the pattern finding task performed during training of the KGE model. Further, implementations of the present disclosure provide a self-monitoring component that can adapt the grouping process depending on a target. A corpus of EHR data, a definition for a cohort to extract from the EHR data and summarize, and a grouping strategy for the creation of, in the example use case, patient groups (personas) are provided as input. In some examples, output includes the optimized dataset (e.g., a patients graph in the example use case), and a report for the content of the graph along with eventual alarms. The entire system is generic and can be tuned to implement different target summary graph depending on the use cases. In the example use case, implementations of the present disclosure leverage patient groups as a central concept, demographic information elements about each patient group, and medical information (conditions, symptoms, vaccinations, etc.) related to each patient group.

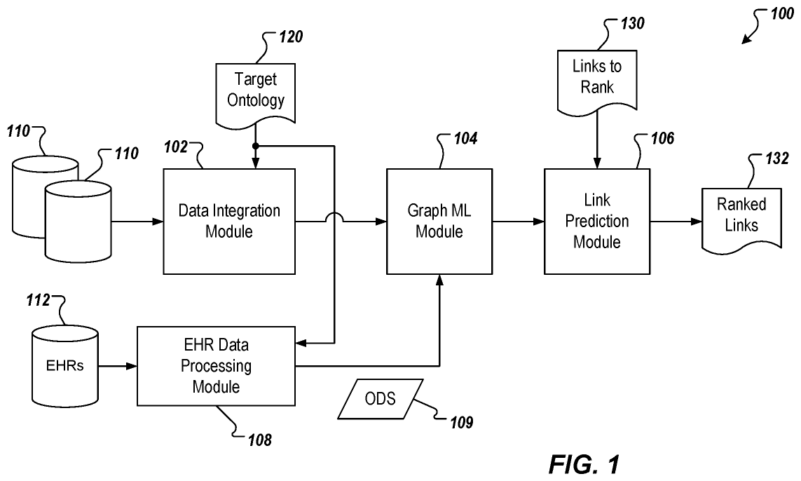

FIG. 1 depicts an example system 100 that can execute implementations of the present disclosure. In the example of FIG. 1, the system 100 includes a data integration module 102, a GML module 104, a link prediction module 106, and an EHR data processing module 108. The GML module 104 trains a KGE model (neural link predictor) using background biology data received from the data integration module 102 and an optimized dataset (ODS) 109 received from the EHR data processing module 108. As described in further detail herein, the ODS 109 is a patients graph that is provided based on patient groups determined from EHR data (i.e., patient medical records). The ODS 109 (patients graph) can be described as a KG that describes relationships between patient groups and concepts (e.g., disease, symptom, drugs, vaccinations, gender, race, birth year, birth decade, etc.) and can represent commonalities between patient groups (e.g., patient groups that share the same race, gender, and/or birth decade). In short, while EHR data is representative of individual patients (patient-level), the patients graph of the present disclosure (e.g., the ODS 109) is representative of patient groups. In some examples, at least a portion of the EHR data is in non-structured, free-text form, while the patients graph is structured.

In some examples, the background biology data is received by the data integration module 102 from one or more databases 110, which processes the background biology data based on a target ontology 120 to provide a biology data KG. In some examples, medical records data is received by the medical record data processing module 108 from one or more EHRs 112. An example EHR includes, but is not limited to, an EHR generated using Synthea, which is provided by The MITRE Corporation, and can be described as a synthetic patient population simulation that is used to generate synthetic patients and that outputs synthetic, but realistic patient data and health records. Each EHR 112 is representative of multiple, individual patients (e.g., patient-level, hundreds of thousands to millions of patients). As described in further detail herein, the medical record data processing module 108 processes the one or more EHRs 112 based on the target ontology 120 to provide the ODS 109. In some examples, the patients graph 109 and the biology data KG of the data integration module 102 are combined into an aggreagate KG, which is fed into the machine learning creating the KGE model.

In some examples, the (trained) KGE model is executed by the link prediction module 106. For example, a set of links 130 is input to the link prediction module 106, which processes each link through the KGE model to generate a score for a link, the score representing a likelihood that the link is factually correct. Here, a link represents a triple (e.g., t′=(s′, p′, o′)). In some examples, the links in the set of links 130 are put in rank order (e.g., most likely to least likely) based on scores to provide a set of ranked links 132 as output of the link prediction module 106.

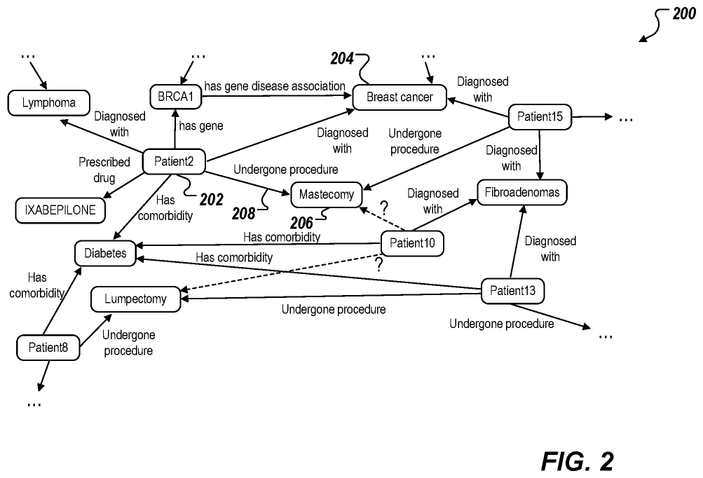

Implementations of the present disclosure are described in further detail herein with non-limiting reference to an example KG representative of the example use case. FIG. 2 depicts an example portion 200 of the example KG, which includes nodes representative of respective entities and edges representative of relationships between entities. The KG can be a multi-relational graph, that is two nodes can be connected by multiple edges with distinct labels. The KG can be denoted as G⊆V×R×V, where I represents the set of nodes and R represents a set of relation types (edges).

The KG is specified by a collection of triples that represent links (also referred to as “facts”) in the data underlying the knowledge graph. As noted above triple can be represented as t=(s, p, o), where s corresponds to a respective node and represents a subject, o corresponds to a respective node and represents an object, and p corresponds to an edge and represents a predicate that describes the relationship between the nodes connected by the edge. Each node can be both a subject and object if it appears in more than one triple.

Each node in the KG (and therefore each subject and object) represents an entity has an entity type. The entity types are dependent on the type of data that the KG represents. For example, the KG shown in FIG. 2 is a KG of a decision support system that can be used to predict whether a patient needs to undergo a certain medical procedure. In this example, the entity type for the node “Patient2” 202 is “patient,” the entity type for the node “Breast cancer” 204 is “disease,” and the entity type for the node “Masectomy” 206 is “surgical procedure.” Since the node “Patient2” 202 is connected to the node “Masectomy” 206 by an edge 208, an example triple that appears in the KG is t=(“patient2”, undergone procedure, “masectomy”) where “patient2” is the object entity, “undergone procedure” is the predicate, and “mastectomy” is the object entity. This triple links patient2 to the surgical procedure mastectomy (i.e., represents the fact that patient2 had a mastectomy).

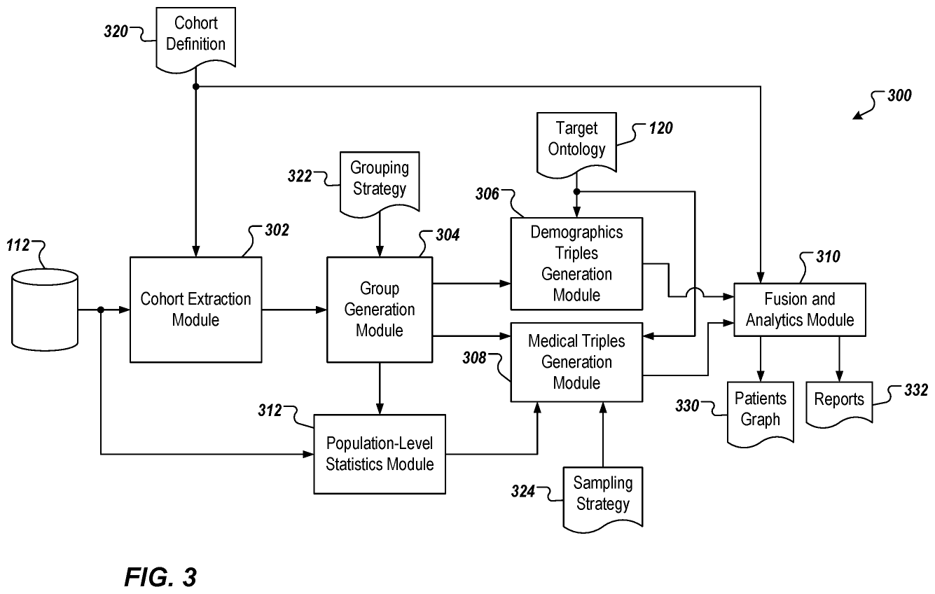

FIG. 3 depicts a conceptual architecture 300 for EHR data summarization in accordance with implementations of the present disclosure. The conceptual architecture 300 can represent internal functionality of the medical record data processing module 108 of FIG. 1.

In the example of FIG. 3, the conceptual architecture 300 includes a cohort extraction module 302, a group generation module 304, a demographics triples generation module 306, a medical triples generation module 308, a fusion and analytics module 310, and a population-level statistics module 312. As input, the conceptual architecture 300 receives the one or more EHRs 112 (medical records data), the target ontology 120, a cohort definition 320, a grouping strategy 322, and a sampling strategy 324. As described in further detail herein, the conceptual architecture 300 provides a patients graph 330 and a report 332 as output. In some examples, the patients graph 330 is or is at least part of the ODS 109 of FIG. 1.

With regard to cohort extraction, the EHR population data set will typically contain records for millions of patients having various medical profiles and histories. Instead of working on all of this data at once, the cohort extraction module 302 generates, in the example use case, patient cohorts, each being a subset of patients having some criteria in common. Those criteria could be any combination of diagnosed condition, demographic aspects (e.g., gender, age, etc.), drug taken, and the like. These criteria are provided in the cohort definition 320. The output of the cohort extraction is a subset of the global EHR population with only data about individuals in scope for the cohort. In this manner, the scale of the data from the EHR can be reduced from millions of patients to thousands of patients, for example. In some examples, a single cohort is extracted for processing of the EHR data, as described herein.

In accordance with implementations of the present disclosure, the group generation module 304 receives the cohort data (subset EHR data) output by the cohort extraction module 302. In the example use case, the cohort data contains EHR data for several patients, where each patient has a particular and unique history. However, the group generation module 304 groups patients into broad profiles, namely the patient groups of the present disclosure, according to some shared criteria. In some examples, a patient group can be described as a generalized description of individual patients that have been grouped based on the shared criteria.

In some examples, the criteria used for grouping are specific to the use case and are defined in the grouping strategy 322. Such criteria can include, for example and without limitation, a shared condition, a shared birth year, a shared gender, and the like. In some examples, one group is created for each combination of criteria. Table 1 provides an example of groups created (e.g., output by the group generation module 304) for the example use case using three grouping criteria each taking two possible values:

TABLE 1
Example Patient Groups

| Group Name | Race | Sex | Birth Decade |
|---|---|---|---|
| Female_White_1980 | White | Female | 1980 |
| Male_Black_1990 | Black | Male | 1990 |
| Female_Black_1990 | Black | Female | 1990 |
| Male_White_1980 | White | Male | 1980 |

In some examples, the choice of criteria to use and definition of the criteria can be determined by a user having expert knowledge. Options available are to include or not include specific criteria, but also adjust one in a particular way or another. For example, in the example of Table 1, it could have been decided to use the exact birth year, or bin the birth year in bins of 5 years instead of 10 years (decade). By doing so, the number of patient groups created would increase. Multiple factors can inform and guide that decision process, which factors can include, for example and without limitation, the size of each patient group and the total number of patient groups created. In some instances, a patient group could be empty. In some examples, a user (e.g., data scientist with expert knowledge) tuning the pipeline would perform some trial-and-error process until a satisfactory result is found.

However, implementations of the present disclosure provide a tuning sub-process to the create target groups. In some examples, the tuning sub-process receives target constraints as input and adjusts patient group creation based on the target constraints. Example target constraints can include significance (e.g., no group under 200 patients) and diversity (e.g., at least ⅓ of all possible groups created). The significance target constraint will aim at finding patient groups that are meaningful in size, where the extremes of a patient group having single person and a patient group having an entire cohort being equally undesirable. The diversity target constraint aims at creating patient groups that are themselves meaningful and representative of the cohort. For example, there should be a good representation of all of the main profiles emerging from the EHR data.

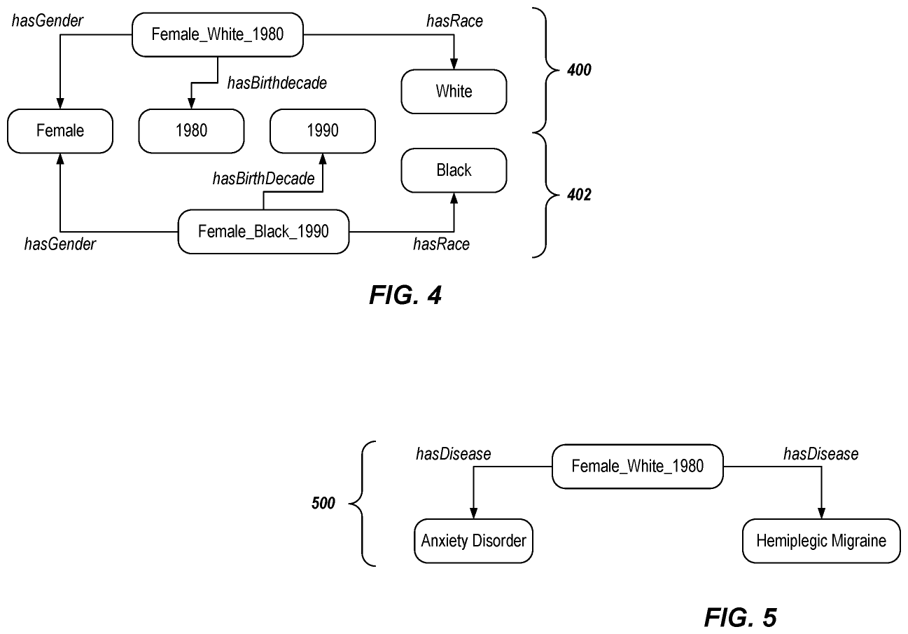

In some implementations, the patient groups are provided to the demographics triples generation module 306, which generates a set of demographics triples for each patient group. FIG. 4 depicts example patient group graphs 400, 402 graphically representing example demographics triples in accordance with implementations of the present disclosure. The patient group graph 400 corresponds to the patient group Female_White_1980 of Table 1 and the patient group graph 402 corresponds to the patient group Female_Black_1990. The patient group graph 400 graphically represent the following example demographics triples:
[Female_White_1980, hasGender, Female]
[Female_White_1980, hasBirthdecade, 1980]
[Female_White_1980, hasRace, White]
   The patient group graph 402 graphically represent the following example demographics triples:
[Female_Black_1990, hasGender, Female]
[Female_Black_1990, hasBirthdecade, 1990]
[Female_Black_1990, hasRace, Black]

The demographics triples enable the GML algorithms to be informed of commonalities between patient groups. For example, in the example of FIG. 4, a commonality between to the patient group Female_White_1980 and the patient group Female_Black_1990 is the object Female.

In some implementations, the population-level statistics module 312 extracts overall statistical information for the patient population represented in the EHR data 112. The output of this process feeds into the generation of the medical triples, for example, to evaluate significance of some data points. Here, the population-level statistics module 312 also takes into account the patient group generated by the group generation module 304. By considering the patient groups, the statistics are calibrated for patient groups instead of individual patients. For example, and without limitation, an example population statistic of “1.5 k individuals take the drug paracetamol” would be determined for an example patient population. However, in the presence of patient groups, this no longer makes sense. Instead, useful statistics will take the form of, for example and without limitation, “Female_white_1980 take the drug Paracetamol.”. In this example, the information loss around the exact number of individuals is already accounted for and mitigated as part of the overall data pipeline.

Which statistics are generated in this process will depend on the implementation use case and in particular the elements within the medical triples. For purposes of non-limiting illustration and as non-exhaustive guidance, population-level statistics that are determined can include: the number of individuals in each patient group, identifiers of drugs taken by each patient group, and identifiers of conditions associated to each group. In some examples, these statistics can be used to determine the conditions and drugs that are to be included in the medical triples, as described in further detail herein.

In some implementations, the patient groups and the population-level statistics are provided to the medical triples generation module 308, which generates a set of medical triples for each patient group. More particularly, the medical triples are generated by extracting the information related to the patient EHRs. This process is aimed at extracting the actual information about what the persons defined in the patient groups take as drugs, have as conditions, took as vaccinations, and the like. The type of information that is extracted will depend on the use case. In some instances, part of the extraction processes will depend on other parts, so it is relevant to implement this specific processing part with a workflow management tool (e.g., Python Luigi). The gist of every process within this processing step will be to determine what is significant to take into account and express as medical triples.

For example, the patient EHR data that is processed is for a cohort that is defined by, for example, a shared diagnosed condition. Consequently, it may not be relevant to add that link, Persona-Condition, to the graph as all of the patient groups (personas) in the cohort will feature that link. In another example, it may be desirable to filter out all of the drugs taken that have nothing to do with the target condition, which defines the cohort. For example, if the cohort focus is defined on patients with an open wound of lower limb, an intake of nervous system drugs may not be relevant.

For purposes of illustration, and without limitation, the extraction of diagnosed condition and drugs is described in further detail herein. However, implementations of the present disclosure can be realized for extraction of any appropriate type of information (e.g., vaccinations). For example, Table 2 below provides a summary of the two types of data discussed in further detail herein and notes how other types of data could fit:

TABLE 2
Example Data Types for Extraction to Medical Triples

| Type of Data | Nature of Record | Approach |
|---|---|---|
| Conditions | Observed | Look for a risk factor to find what is |
| | | statistically significant for the cohort |
| | | considered in terms of conditions and |
| | | co-conditions. |
| Drugs | Prescribed | Look at frequency to assess popular |
| | | and niche usage. |
| Vaccinations | Prescribed | Look at frequency to assess popular |
| | | and niche usage. |
| Observations | Observed | Look for a risk factor to find what is |
| | | statistically significant for the cohort |
| | | considered in terms of biomarkers |
| | | (e.g. weight = particular value). |

In the example data types (and other data types not discussed herein), thresholds can be set. In some examples, the thresholds for respective data types are defined in the sampling strategy 324 that is input to the medical triples generation module 308. In some examples, the thresholds are subject to automated tuning that can be achieved using a set of algorithms optimized for tuning hyper parameters. An example set of algorithms includes, without limitation, estimation of distribution algorithms (EDAs) that will try out several values for the sampling strategy until a satisfying combination is found. Similar to the tuning of the grouping strategy, user input can define constraints in terms of meta-objectives. This could be defined as, for example and without limitation, having between 10 and 20 drugs associated to each patient group, and/or having no more than 50 conditions per patient group. Defining the sampling strategy as such, in terms of outcome on the graph shape, allows for greater control over the shortcomings of GML on graphs with degree imbalance. With this approach, implementations of the present disclosure enable a target outcome for the graph to be defined and reach this outcome following a medical-inspired approach.

With regard to conditions, in the example use case, a condition is something diagnosed to a patient. This can further be specified into being, for example and without limitation, a disease (e.g., flu) or a symptom (e.g., headache), which are accounted for in the target ontology 120. Here, it is to be determined whether a condition is relevant to track as a medical triple or can be ignored. To make this determination, implementations of the present disclosure use an approach traditionally used for identifying comorbidities, but re-purpose this approach for identifying co-occurrences. As discussed above, the EHR dataset that is processed for medical triples is a cohort of individuals all having been diagnosed with the same condition. For all of the other, by definition, co-occurring conditions, a risk factor is determined to express whether this co-occurrence is meaningful. If it is meaningful, a link between the patient group and that condition is kept. If not meaningful, the condition is dropped.

In some examples, an objective of the risk factor is to assess whether a given patient group is more or less at risk to get some other condition Y considering a current condition X. The risk factor is then a ratio of occurrence of a condition for a patient group within the cohort as compared to the same patient group in the general population. This can be generally expressed as, for example and without limitation, “The patient group P in a cohort defined as people having the condition X is Risk (P,X,Y) times more likely to also get the condition Y than the same patient group in the overall population.” A basic mathematical formulation can be provided as Risk (P, X, Y)=Probability (Y, P, X)/Probability (Y, P, population), which is the ratio between the probability of Y onset in persons of type P already impacted with X divided by the similar probability in the general population. Table 3 provides example risk factors for the Female_White_1980 patient group and respective pairs of co-occurring conditions:

TABLE 3
Example Risk Factors

| Patient Group (P) | Condition (X) | Condition (Y) | Risk (P, X, Y) |
|---|---|---|---|
| Female_White_1980 | Hemiplegic | Anxiety | 3.23 |
| | Migraine | Disorder | |
| Female_White_1980 | Hemiplegic | Hemiplegic | 23687.95 |
| | Migraine | Migraine | |
| Female_White_1980 | Hemiplegic | Open wound | 0.27 |
| | Migraine | of lower limb | |

In the example of Table 3, the conditions are diseases. However, it is appreciated that this approach can be used for other conditions, such as symptoms. Further, condition (X) is the condition that the cohort is based on (i.e., all patients diagnosed with hemiplegic migraine are extracted from the original EHR data as a cohort).

In some examples, the risk factors are compared to a threshold risk factor to determine whether a respective co-occurrence is meaningful. In some examples, the threshold is provided in the sampling strategy 324. For example, and without limitation, a threshold of 1.5 can be considered and, for each patient group P, only a link to a condition Y (disease) is provided for, if the risk factor is greater than or equal to 1.5. In the example of Table 3, Anxiety Disorder and Hemiplegic Migraine will be recorded in respective medical triples, whereas Open wound of lower limb will be dropped. FIG. 5 depicts an example patient group graph 500 representing example medical triples in accordance with implementations of the present disclosure. In the example of FIG. 5, the medical triples represented in the patient group graph include:
[Female_White_1980, hasDisease, Anxiety Disorder]
[Female_White_1980, hasDisease, Hemiplegic Migraine]

With regard to drugs, for the processing of drugs a different approach is taken. Contrary to conditions, which is something a person has, drugs are something people take. They take these because of a prescription that is subjective to the person (e.g., doctor) making that prescription. In order to deal with such subjectivity, implementations of the present disclosure use a head-tail approach. Here, in any typical scenario, some drugs will be used very often across all of the individuals mapped to a given patient group and some drugs will be less popular. The head part aims at capturing the former whereas the latter is found in the tail of the drug distribution.

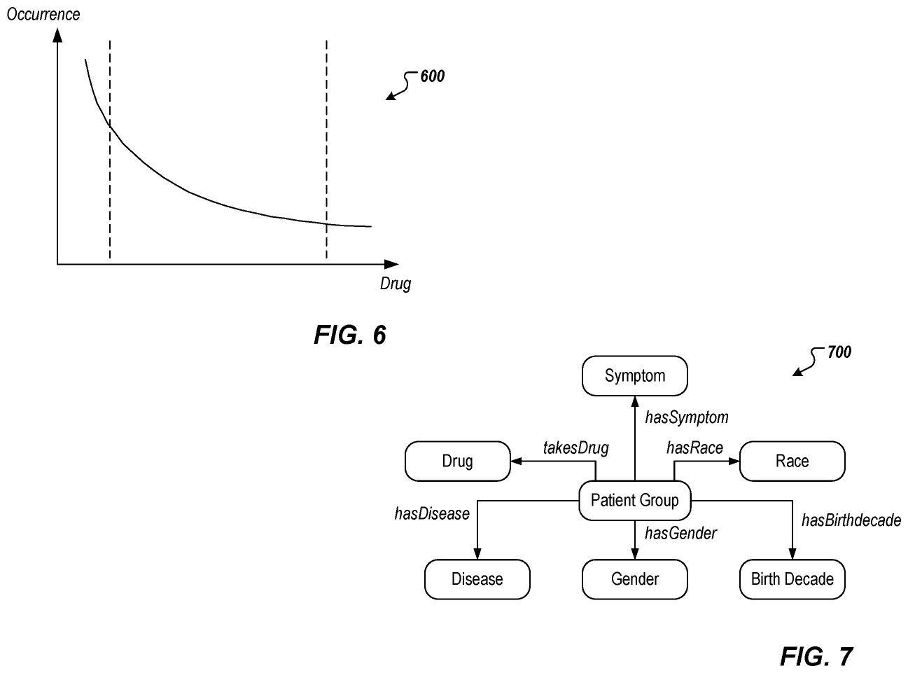

FIG. 6 depicts an example head-tail graph 600 for prescription drugs. Head and tail can be defined in different ways. In some examples, a first 25% percentile and a bottom 75% (meaning equal head and tail in terms of data representation) can be used. Both can be a source of interesting associations that may be worth adding to the graph as well as noise. For example, in any cohort, the drug paracetamol will likely be found in the head as it is a widely prescribed drug with a large spectrum of use. This is, however, a fact bearing little importance for the resulting KG. As another example, in the tail, a not popular or repurposed drug might be found, which turns out to be widely used by that specific cohort. This is a fact that could be easily missed, but which will bring a lot of value to the resulting KG, so it is important to keep it. As a result, a simple approach could include discarding the head (to avoid things like paracetamol edges) and discarding the tail (to cut out noise), which can miss critical data.

In view of this, implementations of the present disclosure apply a filtering approach for the head and tail and discard the rest of the data in the head and tail. In some examples, only drugs that are used at least by a threshold multiple (e.g., 1.5 times) more than in the overall population are kept (e.g., the threshold times being defined in the sampling strategy 324), and ignore drugs that are only used for conditions discarded in the condition filtering (e.g., based on risk factor, as discussed above). The choice of the percentile cut-off and threshold for representation, both part of the strategy, can be as the rest of the strategy tuned using, for example, EDA. For the drugs that remain, triples can be provided for patient groups (e.g., Female_White_1980, takesDrug, Insulin).

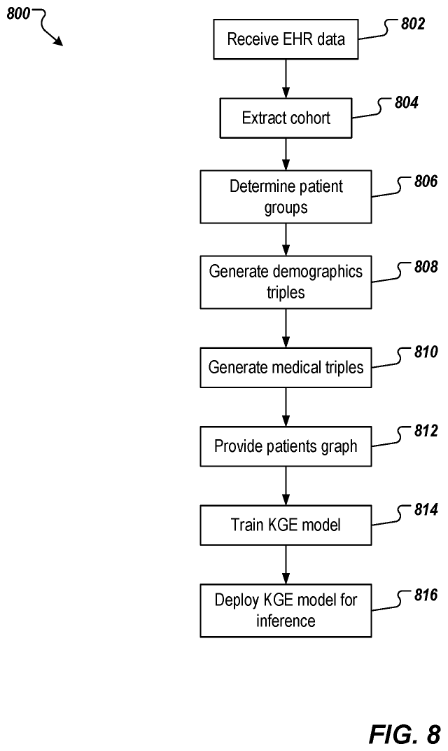

In accordance with implementations of the present disclosure, the demographics triples and the medical triples are provided to the fusion and analytics module 310, which merges the demographics triples and the medical triples into a single KG, provided as the patients graph 330 (e.g., the ODS 109 of FIG. 1). In some examples, merging is achieved by concatenating the triples. In some examples, the patient graph 330 conforms with the target ontology 120. FIG. 7 depicts an example target ontology 700 (e.g., the target ontology 120) in accordance with implementations of the present disclosure. The example target ontology 700 represents triples of:
[Patient Group, hasGender, Gender]
[Patient Group, hasBirthdecade, Birth Decade]
[Patient Group, hasRace, Race]
[Patient Group, hasSymptom, Symptom]
[Patient Group, takesDrug, Drug]
[Patient Group, hasDisease, Disease]

In some examples, each instance of a patient group has less than all of the triples provided for in the target ontology 120. In some examples, each instance of a patient group must include one or more particular triples. In some examples, and as discussed in further detail herein, each instance of a patient group must include at least one condition (e.g., symptom, disease) and must include at least one drug.

In some examples, the fusion and analytics module 310 also provides the report 332, which is based on consistency checking to verify that the final data is coherent. In some examples, the consistency checks include, but are not limited to, validating each patient group as an instance of each of the defined grouping criteria, validating that no patient group has no condition attached to it, and validating that no patient group has no drug attached to it. In some examples, the consistency checks are implemented using a constraint language, such as the Shapes Constraint Language (SHACL). For example, a rule expressing that each instance of a patient group (persona) must be connected to at least one instance of gender is provide in Listing 1:

Listing 1: Example Consistency Check Rule

```
PREFIX sh: <http://www.w3.org/ns/shacl#>
PREFIX xsd: <http://www.w3.org/2001/XMLSchema#>
:NameShape a sh:NodeShape ;
  sh:targetClass :Persona ;
  sh:property [
    sh:path :hasGender ;
    sh:minCount 1 ;
    sh:datatype :Gender
] .
```

In some examples, consistency checks (rules) that fails are reported to one or more users (e.g., an alarm, a notification).

FIG. 8 depicts an example process 800 that can be executed in implementations of the present disclosure. In some examples, the example process 800 is provided using one or more computer-executable programs executed by one or more computing devices.

EHR data is received (802). For example, and as described herein, the cohort extraction module 302 of FIG. 3 receives EHR data 112 that includes medical records of a set of patients. A cohort is extracted (804). For example, and as described herein, the cohort extraction module 302 extracts a patient cohort as a subset of the EHR data, which represents a subset of patients. In some examples, each patient in the subset of patients has one or more criteria (e.g., diagnosed condition, demographic aspects, drug taken, etc.) in common, the criteria being provided in the cohort definition 320.

A set of patient groups is determined (806). For example, and as described herein, the group generation module 304 receives the subset of the EHR data from the cohort extraction module 302 and the grouping strategy 322 and determines a set of patient groups based thereon. In some examples, the group generation module 304 groups patients into patient groups according to some shared criteria, the criteria being defined in the grouping strategy 322. In some examples, a patient group can be described as a generalized description of individual patients that have been grouped based on the shared criteria.

Demographics triples are generated (808). For example, and as described herein, the demographics triples generation module 306 generates a set of demographics triples for each patient group. In some examples, the demographics triples generation module 306 generates demographics triples for a patient group by providing edges (predicates) between the patient group as a subject and one or more demographics as objects. For example, a demographics triple [Female_White_1980, hasGender, Female] is generated by providing a hasGender edge (predict) between Female_White_1980 (subject) and Female (object). Medical triples are generated (810). For example, and as described herein, the medical triples generation module 306 generates a set of medical triples for each patient group. In some examples, medical triples can be generated based on conditions (e.g., diseases, symptoms), drugs, vaccinations, observations, and the like. In some examples, medical triples representative of conditions are determined by determining risk factors for respective co-occurrences of conditions, where conditions of a co-occurrence having a risk factor that meets a threshold risk factor are included in a medical triple. In some examples, medical triples representative of drugs are determined by analysis of head-tail of a drug curve, as described in further detail herein.

A patients graph is provided (812). For example, and as described herein, the fusion and analytics module 310 generates the patients graph 330. In some examples, the patients graph 330 is generated by concatenating the demographics triples and medical triples based on patent group nodes.

A KGE model is trained (814). For example, and as described herein, the graph ML module 104 of FIG. 1 receives the KG from the data integration module 102 and the ODS 109 (e.g., the patients graph 330 of FIG. 3) and trains the KGE model based thereon. The KGE model is deployed for inference (816). For example, and as described herein, the (trained KGE model) is executed by the link prediction module 106 to predict likelihood of links being factually accurate. For example, the set of links 130 is input to the link prediction module 106, which processes each link through the KGE model to generate a score for a link, the score representing a likelihood that the link is factually correct. The links in the set of links 130 are put in rank order (e.g., most likely to least likely) based on scores to provide a set of ranked links 132 as output of the link prediction module 106.

Implementations of the present disclosure can be used, for example and without limitation, in a clinical setting, in which a clinician may be treating a patient and seeking to determine likelihoods that the patient may be diagnosed with one or more conditions, have one or more comorbidities, undergone one or more procedures, and the like. For example, and for purposes of non-limiting illustration, the example portion 200 of the example KG of FIG. 2 can be considered. In this example, the KGE model can be used to predicted likelihoods that Patient 10, who is diagnosed with Fibroadenomas and has comorbidity of Diabetes, has undergone a mastectomy and/or has undergone a Lumpectomy, respectively.

Implementations and all of the functional operations described in this specification may be realized in digital electronic circuitry, or in computer software, firmware, or hardware, including the structures disclosed in this specification and their structural equivalents, or in combinations of one or more of them. Implementations may be realized as one or more computer program products (i.e., one or more modules of computer program instructions encoded on a computer readable medium for execution by, or to control the operation of, data processing apparatus). The computer readable medium may be a machine-readable storage device, a machine-readable storage substrate, a memory device, a composition of matter effecting a machine-readable propagated signal, or a combination of one or more of them. The term “computing system” encompasses all apparatus, devices, and machines for processing data, including by way of example a programmable processor, a computer, or multiple processors or computers. The apparatus may include, in addition to hardware, code that creates an execution environment for the computer program in question (e.g., code that constitutes processor firmware, a protocol stack, a database management system, an operating system, or any appropriate combination of one or more thereof). A propagated signal is an artificially generated signal (e.g., a machine-generated electrical, optical, or electromagnetic signal) that is generated to encode information for transmission to suitable receiver apparatus.

A computer program (also known as a program, software, software application, script, or code) may be written in any appropriate form of programming language, including compiled or interpreted languages, and it may be deployed in any appropriate form, including as a stand-alone program or as a module, component, subroutine, or other unit suitable for use in a computing environment. A computer program does not necessarily correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may be deployed to be executed on one computer or on multiple computers that are located at one site or distributed across multiple sites and interconnected by a communication network.

The processes and logic flows described in this specification may be performed by one or more programmable processors executing one or more computer programs to perform functions by operating on input data and generating output. The processes and logic flows may also be performed by, and apparatus may also be implemented as, special purpose logic circuitry (e.g., an FPGA (field programmable gate array) or an ASIC (application specific integrated circuit)).

Processors suitable for the execution of a computer program include, by way of example, both general and special purpose microprocessors, and any one or more processors of any appropriate kind of digital computer. Generally, a processor will receive instructions and data from a read only memory or a random-access memory or both. Elements of a computer can include a processor for performing instructions and one or more memory devices for storing instructions and data. Generally, a computer will also include, or be operatively coupled to receive data from or transfer data to, or both, one or more mass storage devices for storing data (e.g., magnetic, magneto optical disks, optical disks, solid-state drives (SSDs)). However, a computer need not have such devices. Moreover, a computer may be embedded in another device (e.g., a mobile telephone, a personal digital assistant (PDA), a mobile audio player, a Global Positioning System (GPS) receiver). Computer readable media suitable for storing computer program instructions and data include all forms of non-volatile memory, media and memory devices, including by way of example semiconductor memory devices (e.g., EPROM, EEPROM, and flash memory devices); magnetic disks (e.g., internal hard disks or removable disks); magneto optical disks; and CD ROM and DVD-ROM disks. The processor and the memory may be supplemented by, or incorporated in, special purpose logic circuitry.

To provide for interaction with a user, implementations may be realized on a computer having a display device (e.g., a CRT (cathode ray tube), LCD (liquid crystal display) monitor, OLED (organic light-emitting diode)) for displaying information to the user and a keyboard and a pointing device (e.g., a mouse, a trackball, a touchpad), by which the user may provide input to the computer. Other kinds of devices may be used to provide for interaction with a user as well; for example, feedback provided to the user may be any appropriate form of sensory feedback (e.g., visual feedback, auditory feedback, tactile feedback); and input from the user may be received in any appropriate form, including acoustic, speech, or tactile input.

Implementations may be realized in a computing system that includes a back end component (e.g., as a data server), a middleware component (e.g., an application server), and/or a front end component (e.g., a client computer having a graphical user interface or a Web browser, through which a user may interact with an implementation), or any appropriate combination of one or more such back end, middleware, or front end components. The components of the system may be interconnected by any appropriate form or medium of digital data communication (e.g., a communication network). Examples of communication networks include a local area network (“LAN”) and a wide area network (“WAN”), e.g., the Internet.

The computing system may include clients and servers. A client and server are generally remote from each other and typically interact through a communication network. The relationship of client and server arises by virtue of computer programs running on the respective computers and having a client-server relationship to each other.

While this specification contains many specifics, these should not be construed as limitations on the scope of the disclosure or of what may be claimed, but rather as descriptions of features specific to particular implementations. Certain features that are described in this specification in the context of separate implementations may also be implemented in combination in a single implementation. Conversely, various features that are described in the context of a single implementation may also be implemented in multiple implementations separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems may generally be integrated together in a single software product or packaged into multiple software products.

A number of implementations have been described. Nevertheless, it will be understood that various modifications may be made without departing from the spirit and scope of the disclosure. For example, various forms of the flows shown above may be used, with steps re-ordered, added, or removed. Accordingly, other implementations are within the scope of the following claims.

What is claimed is:

1. A computer-implemented method for providing a knowledge graph embedding (KGE) model for predicting links between entities represented in a knowledge graph (KG), the method comprising:
receiving electronic health record (EHR) data comprising medical records for a set of patients;
defining a set of patient groups from a subset of the EHR data that is representative of a subset of patients of the set of patients, the set of patient groups being defined using a set of criteria providing within a grouping strategy;
generating, for each patient group in the set of patient groups, a set of demographics triples and a set of medical triples, demographics triples in the set of demographics triples comprising one or more links between patient groups and one or more demographics entities, and medical triples in the set of medical triples comprising one or more links between patient groups and one or more medical entities;
providing a patients graph using the set of demographics triples and the set of medical triples;
training a KGE model using a KG and the patients graph to provide a trained KGE model; and
providing the trained KGE model for inference to predict likelihood that a link between entities is factually correct.

2. The computer-implemented method of claim 1, further comprising extracting the subset of the EHR data from the EHR data using a cohort definition that defines at least one cohort criterion.

3. The computer-implemented method of claim 1, wherein generating the set of medical triples comprises:
determining a set of risk factors that represent relative risk of co-occurrence of conditions within the subset of EHR data; and
for each risk factor that meets a threshold risk factor, creating a medical triple representative of the conditions in the set of medical triples.

4. The computer-implemented method of claim 3, wherein a sampling strategy is used to determine the threshold risk factor.

5. The computer-implemented method of claim 1, wherein one or more medical triples represent at least one drug relative to one or more patient groups, the at least one drug being selected for inclusion in the medical triple based on a head-tail analysis of a drug statistics determined from the EHR data based on the patient groups.

6. The computer-implemented method of claim 1, wherein providing a patients graph using the set of demographics triples and the set of medical triples comprising concatenating demographics triples and medical triples.

7. The computer-implemented method of claim 1, wherein demographics triples in the set of demographics triples and medical triples in the set of medical triples are generated based on a target ontology.

8. A system, comprising:
one or more processors; and
a computer-readable storage device coupled to the one or more processors and having instructions stored thereon which, when executed by the one or more processors, cause the one or more processors to perform operations for providing a knowledge graph embedding (KGE) model for predicting links between entities represented in a knowledge graph (KG), the operations comprising:
receiving electronic health record (EHR) data comprising medical records for a set of patients;
defining a set of patient groups from a subset of the EHR data that is representative of a subset of patients of the set of patients, the set of patient groups being defined using a set of criteria providing within a grouping strategy;
generating, for each patient group in the set of patient groups, a set of demographics triples and a set of medical triples, demographics triples in the set of demographics triples comprising one or more links between patient groups and one or more demographics entities, and medical triples in the set of medical triples comprising one or more links between patient groups and one or more medical entities;
providing a patients graph using the set of demographics triples and the set of medical triples;
training a KGE model using a KG and the patients graph to provide a trained KGE model; and
providing the trained KGE model for inference to predict likelihood that a link between entities is factually correct.

9. The system of claim 8, wherein operations further comprise extracting the subset of the EHR data from the EHR data using a cohort definition that defines at least one cohort criterion.

10. The system of claim 8, wherein generating the set of medical triples comprises:
determining a set of risk factors that represent relative risk of co-occurrence of conditions within the subset of EHR data; and
for each risk factor that meets a threshold risk factor, creating a medical triple representative of the conditions in the set of medical triples.

11. The system of claim 10, wherein a sampling strategy is used to determine the threshold risk factor.

12. The system of claim 8, wherein one or more medical triples represent at least one drug relative to one or more patient groups, the at least one drug being selected for inclusion in the medical triple based on a head-tail analysis of a drug statistics determined from the EHR data based on the patient groups.

13. The system of claim 8, wherein providing a patients graph using the set of demographics triples and the set of medical triples comprising concatenating demographics triples and medical triples.

14. The system of claim 8, wherein demographics triples in the set of demographics triples and medical triples in the set of medical triples are generated based on a target ontology.

15. Non-transitory computer-readable storage media coupled to the one or more processors and having instructions stored thereon which, when executed by the one or more processors, cause the one or more processors to perform operations for providing a knowledge graph embedding (KGE) model for predicting links between entities represented in a knowledge graph (KG), the operations comprising:
receiving electronic health record (EHR) data comprising medical records for a set of patients;
defining a set of patient groups from a subset of the EHR data that is representative of a subset of patients of the set of patients, the set of patient groups being defined using a set of criteria providing within a grouping strategy;
generating, for each patient group in the set of patient groups, a set of demographics triples and a set of medical triples, demographics triples in the set of demographics triples comprising one or more links between patient groups and one or more demographics entities, and medical triples in the set of medical triples comprising one or more links between patient groups and one or more medical entities;
providing a patients graph using the set of demographics triples and the set of medical triples;
training a KGE model using a KG and the patients graph to provide a trained KGE model; and
providing the trained KGE model for inference to predict likelihood that a link between entities is factually correct.

16. The non-transitory computer-readable storage media of claim 15, wherein operations further comprise extracting the subset of the EHR data from the EHR data using a cohort definition that defines at least one cohort criterion.

17. The non-transitory computer-readable storage media of claim 15, wherein generating the set of medical triples comprises:
determining a set of risk factors that represent relative risk of co-occurrence of conditions within the subset of EHR data; and
for each risk factor that meets a threshold risk factor, creating a medical triple representative of the conditions in the set of medical triples.

18. The non-transitory computer-readable storage media of claim 17, wherein a sampling strategy is used to determine the threshold risk factor.

19. The non-transitory computer-readable storage media of claim 15, wherein one or more medical triples represent at least one drug relative to one or more patient groups, the at least one drug being selected for inclusion in the medical triple based on a head-tail analysis of a drug statistics determined from the EHR data based on the patient groups.

20. The non-transitory computer-readable storage media of claim 15, wherein providing a patients graph using the set of demographics triples and the set of medical triples comprising concatenating demographics triples and medical triples.

21. The non-transitory computer-readable storage media of claim 15, wherein demographics triples in the set of demographics triples and medical triples in the set of medical triples are generated based on a target ontology.
